(19) United States
(12) Patent Application Publication
(10) Pub. No.: US 2002/0128439 A1
(43) Pub. Date: Sep. 12, 2002

(54) Endoderm, cardiac and neural inducing factors - human frazzled (frzb-1) protein

(75) Inventors: Edward M. De Robertis, Pacific Palisades, CA (US); Tewis Bouwmeester, Heidelberg (DE)

Correspondence Address: Attention : Charles Berman, OPPENHEIMER WOLFF & DONNELLY, 38th Floor, 2029 Century Park East, Los Angeles, CA, 90067-3024, US

(73) Assignee: THE REGENTS OF THE UNIVERSITY OF CALIFORNIA

(21) Appl. No.: 09/903,188

(22) Filed: Jul. 11, 2001

Related U.S. Application Data
(62) Division of application No. 09/552,988, filed on Apr. 21, 2000.
(60) Provisional application No. 60/020,150, filed on Jun. 20, 1996.

(51) Int. Cl. C07K 14/705; C07H 21/04; C12N 15/00

(52) U.S. Cl. 530/350; 536/23.5; 435/320.1

(57) ABSTRACT

Novel proteins have been designated “cerberus” and “frzb-1,” respectively. Cerebus is expressed as a secreted peptide during embryogenesis of the Xenopus embryo, and is expressed specifically in the head organizer region. This new molecule has endodermal, cardiac, and neural tissue inducing activity, that should prove useful in therapeutic, diagnostic, and clinical applications requiring regeneration, differentiation, or repair of these and other tissues. Frzb-1 is a soluble antagonist of growth factors of the Wnt family that acts by binding to Wnt growth factors in the extracellular space. A third novel protein is therm PAPC which promotes the formation of dorsal mesoderm and somites in the embryo.

This application claims the benefit of U.S. Provisional Application No. 60/020,150, filed Jun. 20, 1996.

This invention was made with Government support under grant contract number HD-21502, awarded by the National Institutes of Health. The Government has certain rights in this invention.

### FIELD OF THE INVENTION

The invention generally relates to growth factors, neurotrophic factors, and their inhibitors, and more particularly to several new growth factors with neural, endodermal, and cardiac tissue inducing activity, to complexes and compositions including the factors, and to DNA or RNA coding sequences for the factors. Further, one of the novel growth factors should be useful in tumor suppression gene therapy.

### BACKGROUND OF THE INVENTION

Growth factors are substances, such as polypeptide hormones, which affect the growth of defined populations of animal cells in vivo or in vitro, but which are not nutrient substances. Proteins involved in the growth and differentiation of tissues may promote or inhibit growth, and promote or inhibit differentiation, and thus the general term “growth factor” includes cytokines, trophic factors, and their inhibitors.

Widespread neuronal cell death accompanies normal development of the central and peripheral nervous systems. Studies of peripheral target tissues during development have shown that neuronal cell death results from the competition among neurons for limiting amounts of survivor factors (“neurotrophic factors”). The earliest identified of these, nerve growth factor (“NGF”), is the most fully characterized and has been shown to be essential for the survival of sympathetic and neural crest-derived sensory neurons during early development of both chick and rat.

One family of neurotropic factors are the Wnts, which have dorsal axis-inducing activity. Most of the Wnt proteins are bound to cell surfaces. (See, e.g., Sokol et al., Science, 249, pp. 561-564, 1990.) Dorsal axis-inducing activity in xenopus embryos by one member of this family (Xwnt-8) was described by Smith and Harland in 1991, Cell, 67, pp. 753-765. The authors described using RNA injections as a strategy for identifying endogenous RNAs involved in dorsal patterning to rescue dorsal development in embryos that were ventralized by UV irradiation.

Another member of the growth and neurotropic factor family was subsequently discovered and described by Harland and Smith, which they termed “noggin.” (Cell, 70, pp. 829-840 (1992).) Noggin is a good candidate to function as a signaling molecule in Nieuwkoop's center, by virtue of its maternal transcripts, and in Spemann's organizer, through its zygotic organizer-specific expression. Besides noggin, other secreted factors may be involved in the organizer phenomenon.

Another Xenopus gene designated “chordin ” that begins to be expressed in Spemann's organizer and that can completely rescue axial development in ventralized embryos was described by Sasai et al., Cell, 79, pp. 779-790, 1994. In addition to dorsalizing mesoderm, chordin has the ability to induce neural tissue and its activities are antagonized by Bone Morphogenetic Protein-4 (Sasai et al., Nature, 376, pp. 333-336, 1995).

Therefore, the dorsal lip or Spemann's organizer of the Xenopus embryo is an ideal tissue for seeking novel growth and neurotrophic factors. New growth and neurotrophic factors are useful agents, particularly those that are secreted due to their ability to be used in physiologically active, soluble forms because these factors, their receptors, and DNA or RNA coding sequences therefore and fragments thereof are useful in a number of therapeutic, clinical, research, diagnostic, and drug design applications.

### SUMMARY OF THE INVENTION

In one aspect of the present invention, the sequence of the novel peptide that can be in substantially purified form is shown by SEQ ID NO: 1. The Xenopus derived SEQ ID NO: 1 has been designated “cerberus,” and this peptide is capable of inducing endodermal, cardiac, and neural tissue development in vertebrates when expressed. The nucleotide sequence which, when expressed results in cerberus, is illustrated by SEQ ID NO: 2. Since peptides of the invention induce endodermal, cardiac, and neural tissue differentiation in vertebrates, they should be able to be prepared in physiologically active form for a number of therapeutic, clinical, and diagnostic applications.

Cerberus was isolated during a search for molecules expressed specifically in Spemann's organizer containing a secretory signal sequence. In addition to cerberus, two other novel cDNAs were identified.

The Xenopus derived peptide that can be deduced from SEQ ID NO: 3 encodes a novel protein we had earlier designated as “frazzled,” a secreted protein of 318 amino acids that has dorsalizing activity in Xenopus embryos. We now designate the novel protein as “frzb-1.” The gene for frzb-1 is expressed in many adult tissues of many animals, three of the cDNAs (Xenopus, mouse, and human) have been cloned by us. The accession numbers for the Xenopus, mouse, and human frzb-1 cDNA sequences of the gene now designated frzb-1 are U68059, U68058, and U68057, respectively. Frzb-1 has some degree of sequence similarity to the Drosophila gene frizzled which has been shown to encode a seven-transmembrane protein that can act both as a signalling and as a receptor protein (Vinson et al., Nature, 338, pp. 263-264, 1989; Vinson and Adler, Nature, 329, pp. 549-551, 1987). Vertebrate homologues of Frizzled have been isolated and they too were found to be anchored to the cell membrane by seven membrane spanning domains (Wang et al., J. Biol. Chem., 271, pp. 4468-4476, 1996). Frzb-1 differs from the frizzled proteins in that it is an entirely soluble, diffusible secreted protein and therefore suitable as a therapeutic agent. The nucleotide sequence derived from Xenopus that, when expressed, results in frzb-1 protein is illustrated by SEQ ID NO: 4. The frzb-1 protein derived from mouse is shown as SEQ ID NO: 7, while the mouse frzb-1 nucleotide sequence is SEQ ID NO: 8. The human derived frzb-1 protein is illustrated by SEQ ID NO: 9, and the human frzb-1 nucleotide sequence is SEQ ID NO: 10.

Frzb-1 is an antagonist of Wnts in vivo, and thus is believed to find utility as a tumor suppressor gene, since overexpressed Wnt proteins cause cancer. Frzb-1 may also be a useful vehicle for solubilization and therapeutic delivery of wnt proteins complexed with it.

The final cDNA isolated containing a signal sequence results in a peptide designated Paraxial Protocadherin (PAPC). The cDNA for PAPC is a divergent member of the cadherin multigene family. PAPC is most related to protocadherin 43 reported by Sano et al., The EMBO J., 12, pp. 2249-2256, 1993. As shown in SEQ ID NO: 5, the PAPC gene encodes a transmembrane protein of 896 amino acids, of which 187 are part of an intracellular domain. PAPC is a cell adhesion molecule, and microinjection of PAPC mRNA constructs into Xenopus embryos suggest that PAPC acts as a molecule involved in mesoderm differentiation. A soluble form of the PAPC extracellular domain is able to block muscle and mesoderm formation in Xenopus embryos. The nucleotide sequence encoding Xenopus PAPC is provided in SEQ ID NO: 6.

Cerberus, frzb-1, or PAPC or fragments thereof (which also may be synthesized by in vitro methods) may be fused (by recombinant expression or in vitro covalent methods) to an immunogenic polypeptide and this, in turn, may be used to immunize an animal in order to raise antibodies against the novel proteins. Antibodies are recoverable from the serum of immunized animals. Alternatively, monoclonal antibodies may be prepared from cells from the immunized animal in conventional fashion. Immobilized antibodies are useful particularly in the diagnosis (in vitro or in vivo) or purification of cerberus, frzb-1, or PAPC.

Substitutional, deletional, or insertional mutants of the novel polypeptides may be prepared by in vitro or recombinant methods and screened for immuno-crossreactivity with cerberus, frzb-1, or PAPC and for cerberus antagonist or agonist activity.

Cerberus or frzb-1 also may be derivatized in vitro in order to prepare immobilized and labelled proteins, particularly for purposes of diagnosis of insufficiencies thereof, or for affinity purification of antibodies thereto.

Among applications for the novel proteins are tissue replacement therapy and, because frzb-1 is an antagonist of Wnt signaling, tumor suppression therapies. The cerberus receptor may define a novel signalling pathway. In addition, frzb-1 could permit the isolation of novel members of the Wnt family of growth factors.

### BRIEF DESCRIPTION OF THE DRAWINGS

FIG. 1 illustrates the amino acid sequence (SEQ ID NO: 1) of the FIG. 2 cDNA clone for cerberus;

FIG. 2 illustrates a cDNA clone (SEQ ID NO: 2) for cerberus derived from Xenopus. Sense strand is on top (5′ to 3′ direction) and the antisense strand on the bottom line (in the opposite direction);

FIGS. 3 and 4 show the amino acid and nucleotide sequence, respectively, of full-length frzb-1 from Xenopus (SEQ ID NOS: 3 and 4);

FIGS. 5 and 6 show the amino acid and nucleotide sequence, respectively, of full-length PAPC from Xenopus (SEQ ID NOS: 5 and 6);

FIGS. 7 and 8 show the amino acid and nucleotide sequence, respectively, of full-length frzb-1 from mouse (SEQ ID NOS: 7 and 8); and

FIGS. 9 and 10 show the amino acid and nucleotide sequence, respectively, of full-length frzb-1 from human (SEQ ID NOS: 9 and 10).

### DETAILED DESCRIPTION OF THE PREFERRED EMBODIMENTS

Among the several novel proteins and their nucleotide sequences described herein, is a novel endodermal, cardiac, and neural inducing factor in vertebrates that we have named “cerberus.” When referring to cerberus, the present invention also contemplates the use of fragments, derivatives, agonists, or antagonists of cerberus molecules. Because cerberus has no homology to any reported growth factors, it is proposed to be the founding member of a novel family of growth factors with potent biological activities, which may be isolated using SEQ ID NO: 2.

The amphibian organizer consists of several cell populations with region-specific inducing activities. On the basis of morphogenetic movements, three very different cell populations can be distinguished in the organizer. First, cells with crawling migration movements involute, fanning out to form the prechordal plate. Second, cells involute through the dorsal lip driven by convergence and extension movements, giving rise to the notochord of the trunk. Third, involution ceases and the continuation of mediolateral intercalation movements leads to posterior extension movements and to the formation of the tail notochord and of the chordoneural hinge. The three cell populations correspond to the head, trunk, and tail organizers, respectively.

The cerberus gene is expressed at the right time and place to participate in cell signalling by Spemann's organizer. Specifically, cerberus is expressed in the head organizing region that consists of crawling-migrating cells. The cerberus expressing region corresponds to the prospective foregut, including the liver and pancreas anlage, and the heart mesoderm. Cerberus expression is activated by chordin, noggin, and organizer-specific homeobox genes.

Our studies were conducted in early embryos of the frog Xenopus laevis. The frog embryo is well suited to experiments, particularly experiments pertaining to generating and maintaining regional differences within the embryo for determining roles in tissue differentiation. It is easy to culture embryos with access to the embryos even at very early stages of development (preceding and during the formation of body pattern and differentiation) and the embryos are large. The initial work with noggin and chordin also had been in Xenopus embryos, and, as predicted, was highly conserved among vertebrates. Predictions based on work with Xenopus as to corresponding human noggin were proven true and the ability to clone the gene for human noggin was readily accomplished. (See the description of Xenopus work and cloning information in PCT application, published Mar. 17, 1994, WO 9 405 800, and the subsequent human cloning based thereon in the PCT application, also published Mar. 17, 1994, as WO 9 405 791.)

### CLONING

The cloning of cerberus, frzb-1, and PAPC resulted from a comprehensive screen for cDNAs enriched in Spemann's organizer. Subtractive differential screening was performed as follows. In brief, poly A⁺ RNA was isolated from 300 dorsal lip and ventral marginal zone (VMZ) explants at stage 10½. After first strand cDNA synthesis approximately 70-80% of common sequences were removed by substraction with biotinylated VMZ poly A⁺ RNA prepared from 1500 ventral gastrula halves. For differential screening, duplicate filters (2000 plaques per 15 cm plate, a total of 80,000 clones screened) of an unamplified oriented dorsal lip library were hybridized with radiolabeled dorsal lip or VMZ cDNA. Putative organizer-specific clones were isolated, grouped by sequence analysis from the 5′ end and whole-mount in situ hybridization, and subsequently classified into known and new dorsal-specific genes. Rescreening of the library (100,000 independent phages) with a cerberus probe resulted in the isolation of 45 additional clones, 31 of which had similar size as the longest one of the 11 original clones indicating that they were presumably full-length cDNAs. The longest cDNAs for cerberus, frzb-1, and PAPC were completely sequenced.

To explore the molecular complexity of Spemann's organizer we performed a comprehensive differential screen for dorsal-specific cDNAs. The method was designed to identify abundant cDNAs without bias as to their function. As shown in Table 1, five previously known cDNAs and five new ones were isolated, of which three (expressed as cerberus, frzb-1, and PAPC, respectively) had secretory signal sequences.

TABLE 1

| | |
|---|---|
| Gene Product | No. of Isolates |

Previously Known Genes

| | | |
|---|---|---|
| Chordin | novel secreted protein | 70 |
| Goosecoid | homeobox gene | 3 |
| Pintallavis/XFKH-1 | forkhead/transcription factor | 2 |
| Xnot-2 | homeobox gene | 1 |
| Xlim-1 | homeobox gene | 1 |
| New Genes | | |
| Cerberus | novel secreted protein | 11 |
| PAPC | cadherin-like/transmembrane | 2 |
| Frzb-1 | novel secreted protein | 1 |
| Sox-2 | sry/transcription factor | 1 |
| Fkh-like | forkhead/transcription factor | 1 |

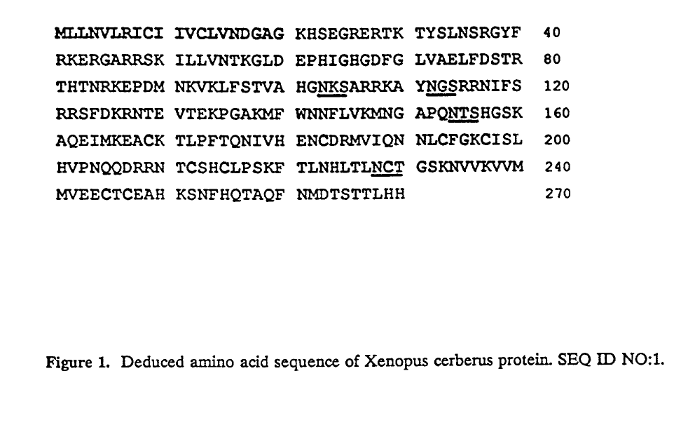

The most abundant dorsal-specific cDNA was chordin (chd), with 70 independent isolates. The second most abundant cDNA was isolated 11 times and named cerberus (after a mythological guardian dog with multiple heads). The cerberus cDNA encodes a putative secreted polypeptide of 270 amino acids, with an amino terminal hydrophobic signal sequence and a carboxy terminal cysteine-rich region (FIG. 1). Cerberus is expressed specifically in the head organizer region of the Xenopus embryo, including the future foregut.

An abundant mRNA found in the dorsal region of the Xenopus gastrula encodes the novel putative secreted protein we have designated as cerberus. Cerberus mRNA has potent inducing activity in Xenopus embryos, leading to the formation of ectopic heads. Unlike other organizer-specific factors, cerberus does not dorsalize mesoderm and is instead an inhibitor of trunk-tail mesoderm. Cerberus is expressed in the anterior-most domain of the gastrula including the leading edge of the deep layer of the dorsal lip a region that, as shown here, gives rise to foregut and midgut endoderm. Cerberus promotes the formation of cement gland, olfactory placodes, cyclopic eyes, forebrain, and duplicated heart and liver (a foregut derivative). Because the pancreas is also derived from this foregut region, it is likely that cerberus induces pancreas in addition to liver. The expression pattern and inducing activities of cerberus suggest a role for a previously neglected region of the embryo, the prospective foregut endoderm, in the induction of the anterior head region of the embryo.

Turning to FIG. 1, Xenopus cerberus encodes a putative secreted protein transiently expressed during embryogenesis and the deduced amino acid sequence of Xenopus cerberus is shown. The signal peptide sequence and the nine cysteine residues in the carboxy-terminus are indicated in bold. Potential N-linked glycosylation sites are underlined. In database searches the cerberus protein showed limited similarity only to the mammalian Dan protein, a possible tumor suppressor proposed to be a DNA-binding protein.

Cerberus appears to be a pioneer protein, as its amino acid sequence and the spacing of its 9 cysteine residues were not significantly similar to other proteins in the databases (NCBI-Gen Bank release 93.0). We conclude that the second most abundant dorsal-specific cDNA encodes a novel putative secreted factor, which should be the founding member of a novel family of growth factors active in cell differentiation.

Cerberus Demarcates an Anterior Organizer Domain. Cerberus mRNA is expressed at low levels in the unfertilized egg, and zygotic transcripts start accumulating at early gastrula. Expression continues during gastrula and early neurula, rapidly declining during neurulation. Importantly, cerberus expression starts about one hour after that of chd, suggesting that cerberus could act downstream of the chd signal.

Whole-mount in situ hybridizations reveal that expression starts in the yolky endomesodermal cells located in the deep layer of the organizer. The cerberus domain includes the leading edge of the most anterior organizer cells and extends into the lateral mesoderm. The leading edge gives rise to liver, pancreas, and foregut in its midline, and the more lateral region gives rise to heart mesoderm at later stages of development.

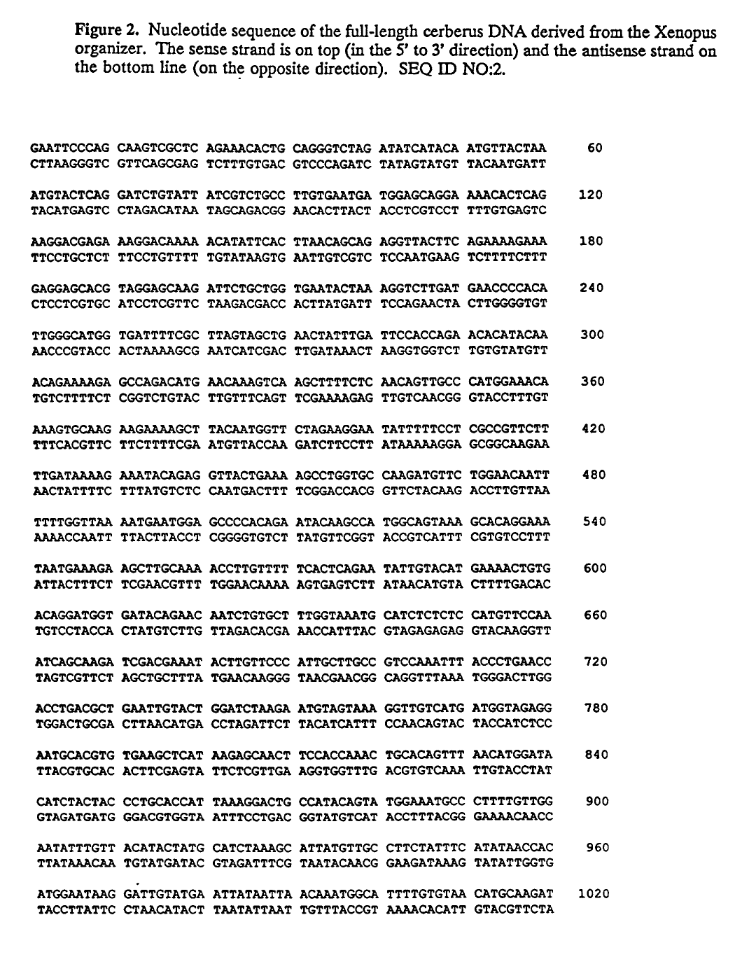

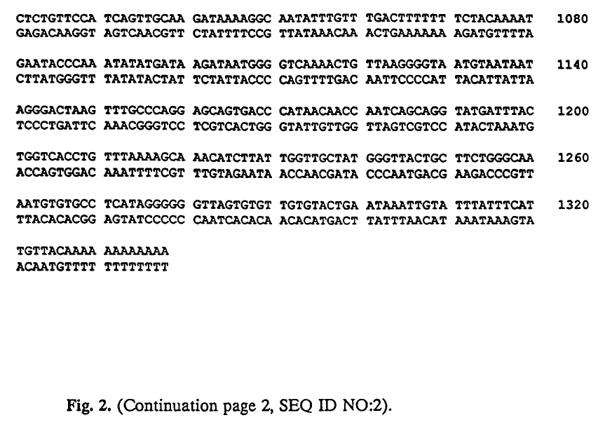

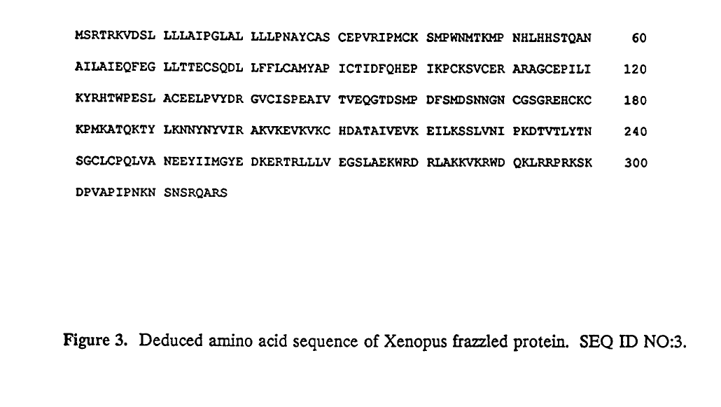

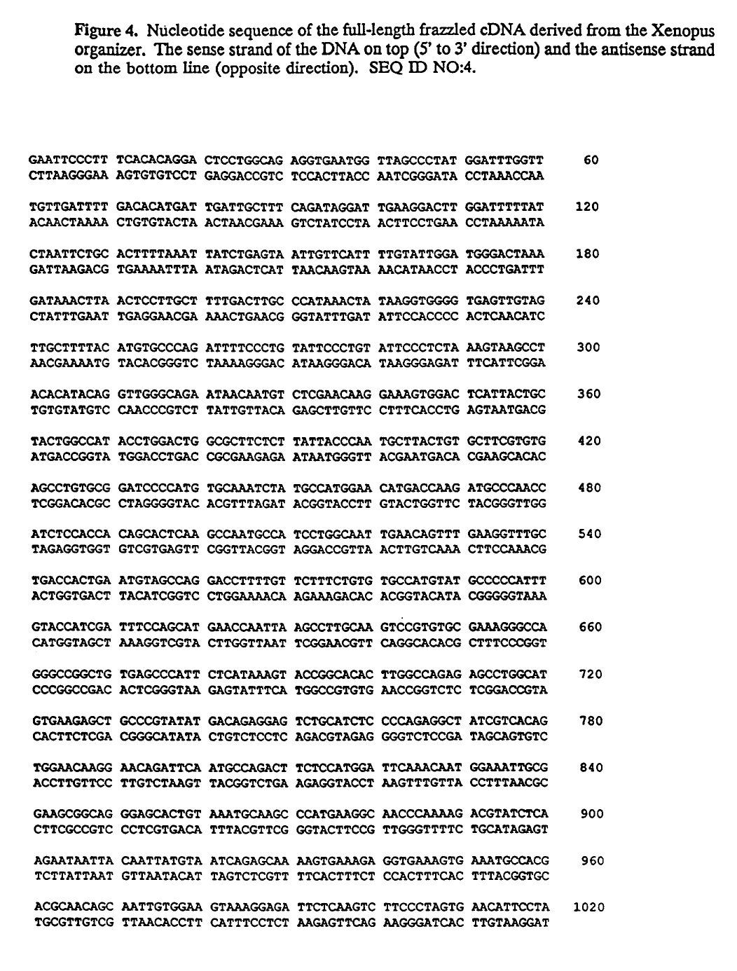

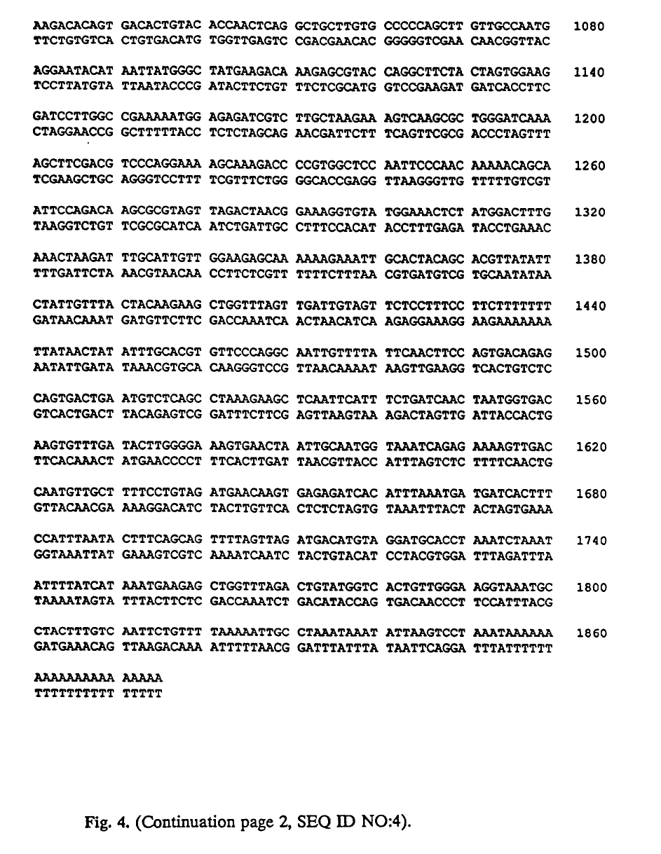

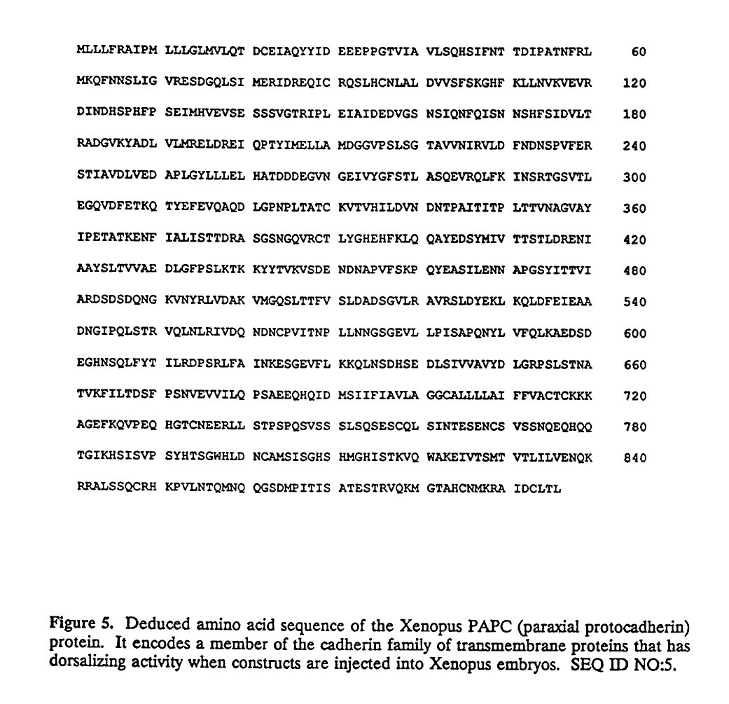

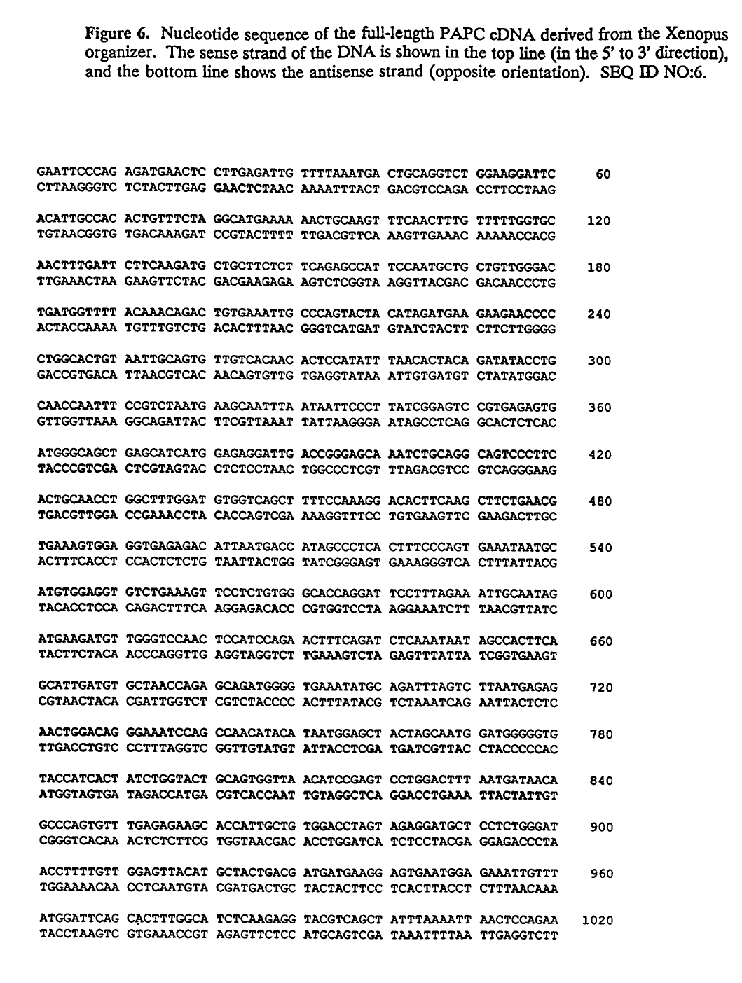

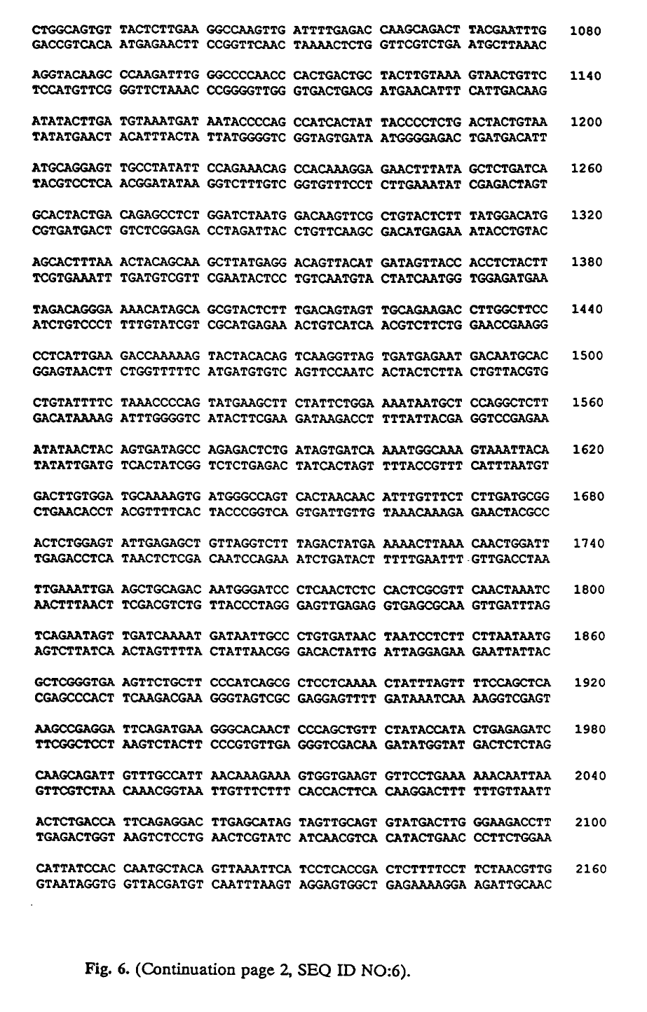

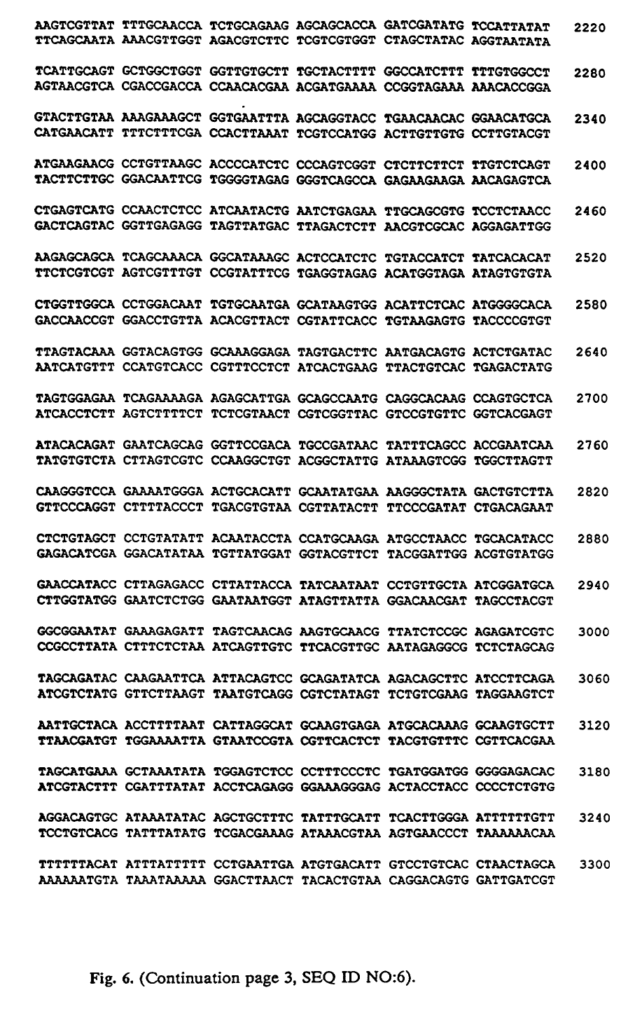

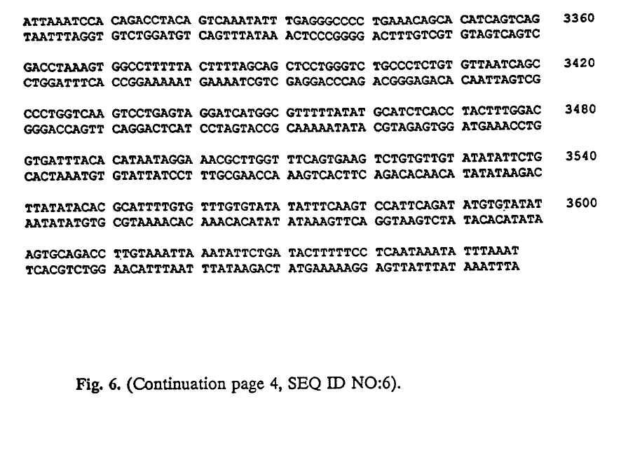

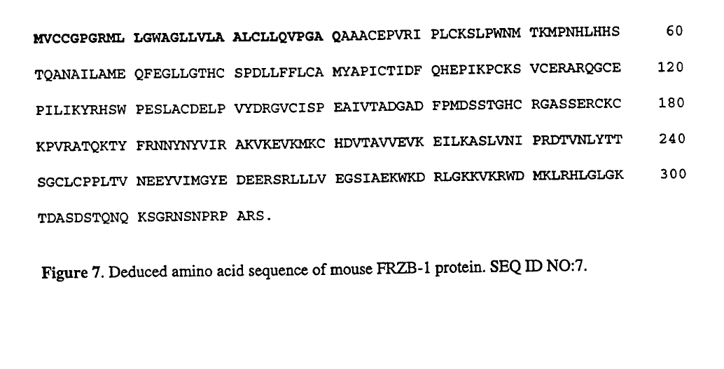

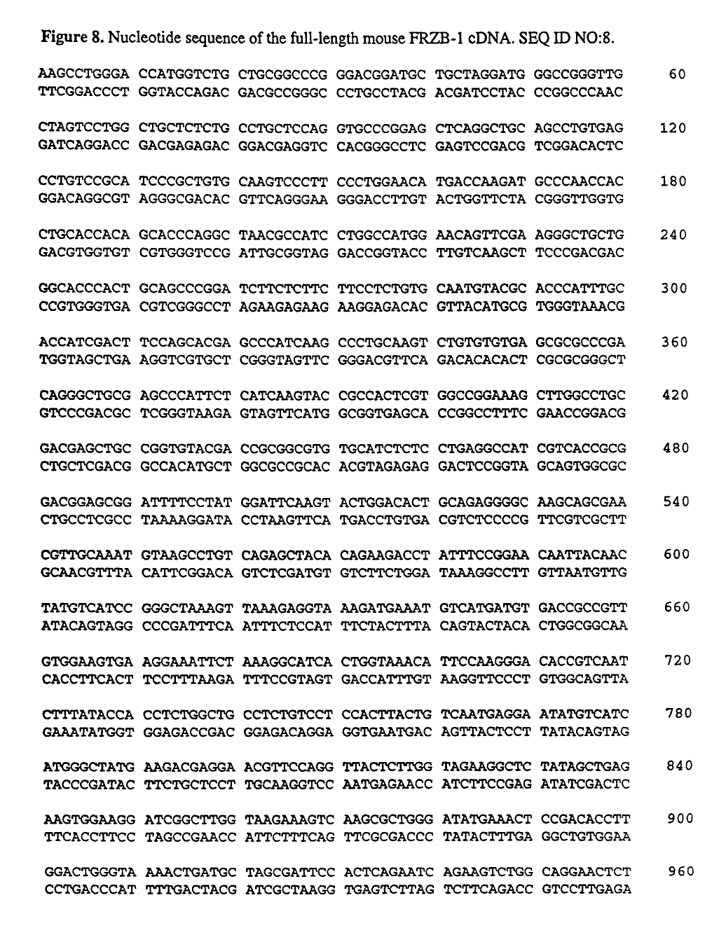

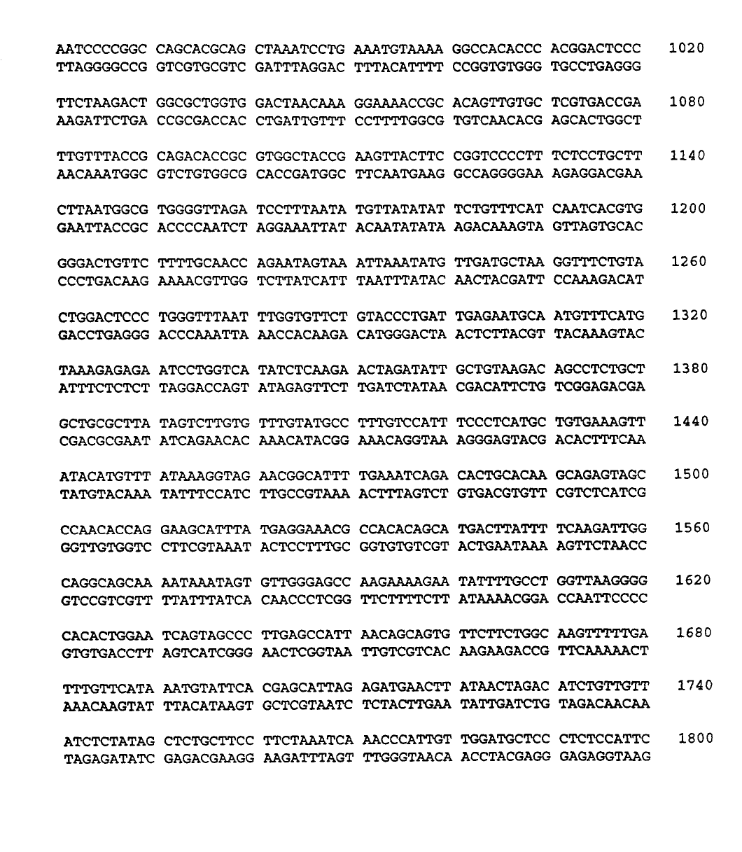

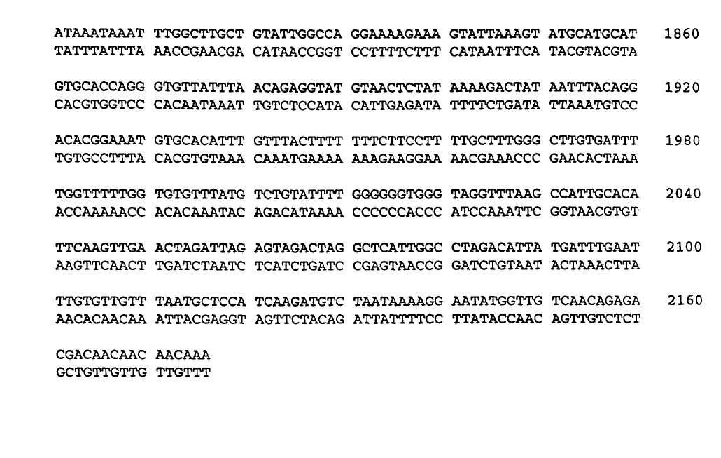

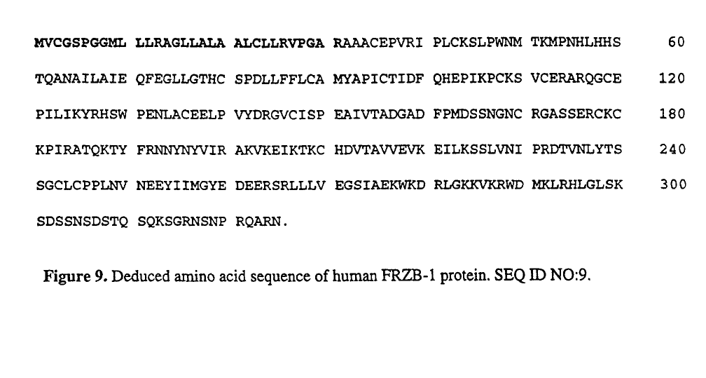

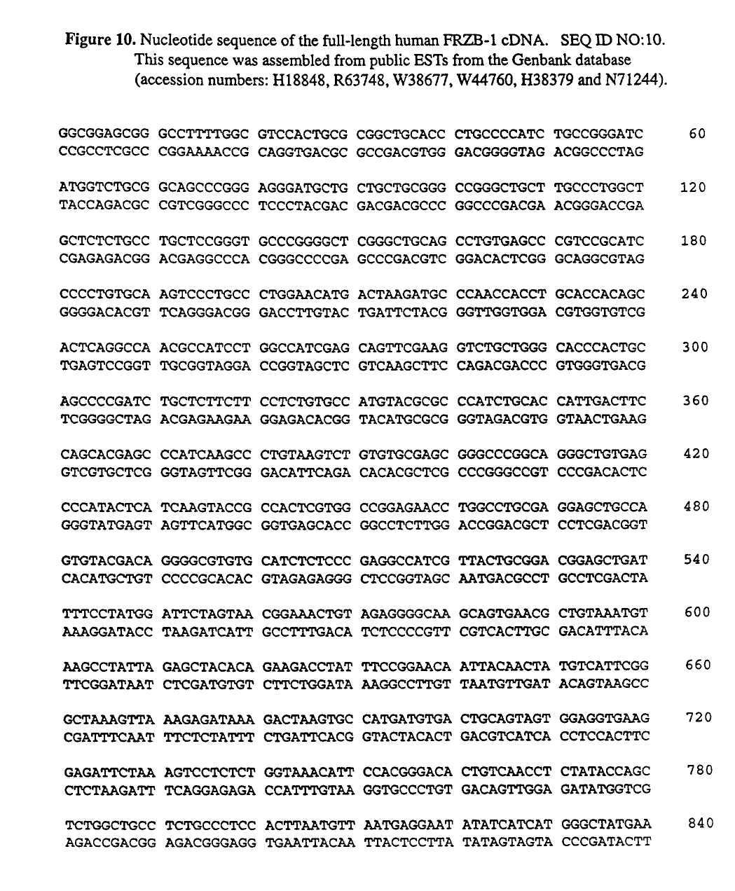

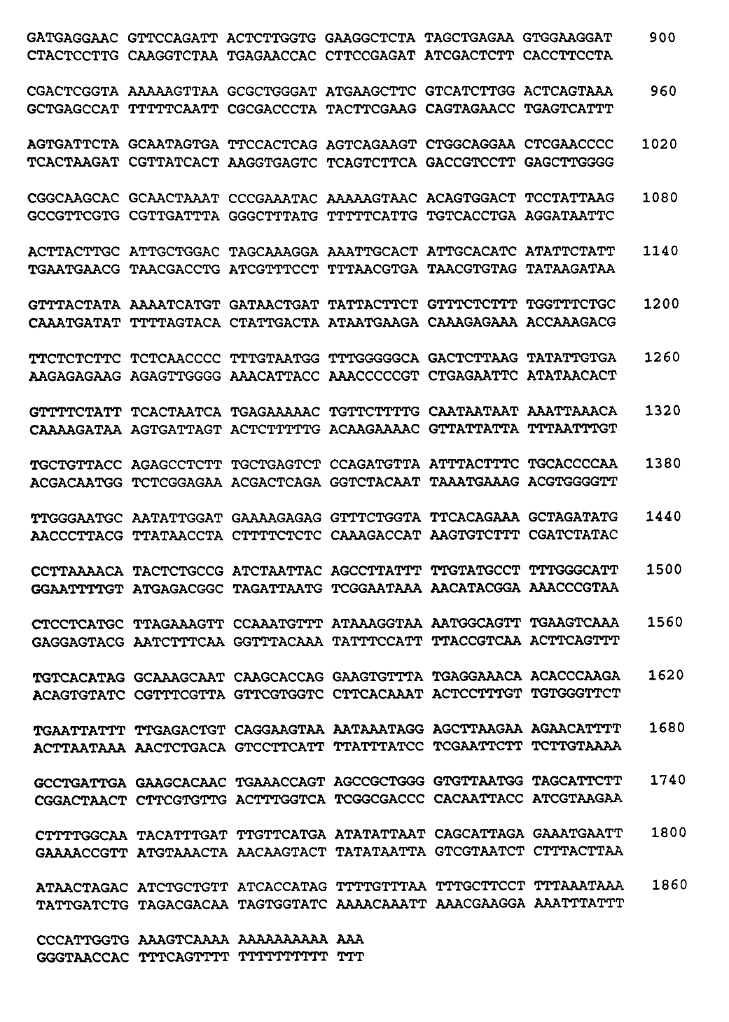

FIG. 2 sets out the sequence of a full length Xenopus cDNA for cerberus.

This entirely new molecule has demonstrated physiological properties that should prove useful in therapeutic, diagnostic, and clinical applications that require regeneration, differentiation, or repair of tissues, such wound repair, neuronal regenerational or transplantation, supplementation of heart muscle differentiation, differentiation of pancreas and liver, and other applications in which cell differentiation processes are to be induced.

The second, novel, secreted protein we have discovered is called “frzb-1,” which was shown to be a secreted protein in Xenopus oocyte microinjection experiments. Thus it provides a natural soluble form of the related extracellular domains of Drosophila and vertebrate frizzled proteins. we propose that the latter proteins could be converted into active soluble forms by introducing a stop codon before the first transmembrane domain. We have noted that the cysteine-rich region of frzb-1 and frizzled contains some overall structural homology with Wnt proteins using the Profile Search homology program (Gribskov, Meth. Enzymol., 183, pp. 146-159, 1990). This had raised the interesting possibility that frzb-1 could interact directly with Wnt growth factors in the extracellular space. This was because we had found that when microinjected into Xenopus embryos, frzb-1 constructs have moderate dorsalizing activity, leading to the formation of embryos with enlarged brain and head, and shortened truck. Somatic muscle differentiation, which requires Xwnt-8, was inhibited. In the case of frzb-1, an attractive hypothesis, suggested by the structural homologies, was that it may act as an inhibitor of Wnt-8, a growth factor that has ventralizing activity in the Xenopus embryo (Christian and Moon, Genes Dev., 7, pp. 13-28, 1993). We have shown that frzb-1 can interact with Xwnt-8 and Wnt-1, and it is expected that it could also interact with other members of the wnt family of growth factors, of which at least 15 members exist in mammals. In addition, a possible interaction with Wnts was suggested by the recent discovery that dishevelled, a gene acting downstream of wingless, has strong genetic interaction with frizzled mutants in Drosophila (Krasnow et al., Development, 121, pp. 4095-4102, 1995). This possibility has been explored in depth (Leyns et al., Cell, 88, pp. 747-756, Mar. 21, 1997), because a soluble antagonist of the Wnt family of proteins is expected to be of great therapeutic value. Examples 1 and 2 illustrate tests that show antagonism of Xwnt-8 by binding to frzb-1.

Vertebrate homologues of Frizzled have been isolated and they too are anchored to the cell membrane by seven membrane spanning domains (Wang et al., J. Biol. Chem., 271, pp. 4468-4476, 1996). Frzb-1 differs from the frizzled proteins in that it is an entirely soluble, diffusible secreted protein and therefore suitable as a therapeutic agent. The nucleotide sequence that when expressed results in frzb-1 protein is illustrated by SEQ ID NO: 4.

SEQ ID NO: 4 corresponds to the Xenopus homolog, but by using it in BLAST searches (and by cloning mouse frzb-1) we had been able to assemble the sequence of the entire mature human frzb-1 protein, SEQ ID NO: 9. Indeed, human frzb-1 is encoded in six expressed sequence tags (ESTs) available in Genebank. The human frzb-1 sequence can be assembled by overlapping in the 5′ to 3′ direction the ESTs with the following accession numbers in Genebank: H18848, R63748, W38677, W44760, H38379, and N71244. No function had yet been assigned to these EST sequences, but we believe and thus propose here that human frzb-1 will have similar functions in cell differentiation to those described above for Xenopus frzb-1. The nucleotide sequence of human frzb-1 is shown in SEQ ID NO: 10. The mouse frzb-1 protein and nucleotide sequences are provided by SEQ ID NOS: 7 and 8, respectively.

In particular, we believe that frzb-1 will prove useful in gene therapy of human cancer cells. In this rapidly developing field, one approach is to introduce vectors expressing anti-sense sequences to block expression of dominant ocogenes and growth factor receptors. Another approach is to produce episomal vectors that will replicate in human cells in a controlled fashion without transforming the cells. For an example of the latter (an episomal expression vector system for human gene therapy), reference is made to U.S. Pat. No. 5,624,820, issued Apr. 29, 1997, inventor Cooper.

Gene therapy now includes uses of human tumor suppression genes. For example, U.S. Pat. No. 5,491,064, issued Feb. 13, 1996, discloses a tumor suppression gene localized on chromosome 11 and described as potentially useful for gene therapy in cancers deleted or altered in their expression of that gene. Frzb-1 maps to chromosome 2q31-33 and loss of one copy of the 2q31-33 and loss of one copy of the 2q arm has been observed with high incidence in lung carcinomas, colo-rectal carcinomas, and neuroblastomas, which has lead to the proposal that the 2q arm carries a tumor suppressor gene. We expect frzb to be a tumor suppressor gene, and thus to be useful in tumor suppression applications.

A number of applications for cerberus and frzb-1 are suggested from their pharmacological (biological activity) properties.

For example, the cerberus and frzb-1 cDNAs should be useful as a diagnostic tool (such as through use of antibodies in assays for proteins in cell lines or use of oligonucleotides as primers in a PCR test to amplify those with sequence similarities to the oligonucleotide primer, and to determine how much of the novel protein is present).

Cerberus, of course, might act upon its target cells via its own receptor. Cerberus, therefore, provides the key to isolate this receptor. Since many receptors mutate to cellular oncogenes, the cerberus receptor should prove useful as a diagnostic probe for certain tumor types. Thus, when one views cerberus as ligand in complexes, then complexes in accordance with the invention include antibody bound to cerberus, antibody bound to peptides derived from cerberus, cerberus bound to its receptor, or peptides derived from cerberus bound to its receptor or other factors. Mutant forms of cerberus, which are either more potent agonists or antagonists, are believed to be clinically useful. Such complexes of cerberus and its binding protein partners will find uses in a number of applications.

Practice of this invention includes use of an oligonucleotide construct comprising a sequence coding for cerberus or frzb-1 and for a promoter sequence operatively linked in a mammalian or a viral expression vector. Expression and cloning vectors contain a nucleotide sequence that enables the vector to replicate in one or more selected host cells. Generally, in cloning vectors this sequence is one that enables the vector to replicate independently of the host chromosomes, and includes origins of replication or autonomously replicating sequences. The well-known plasmid pBR322 is suitable for most gram negative bacteria, the 2μ plasmid origin for yeast and various viral origins (SV40, polyoma, adenovirus, VSV or BPV) are useful for cloning vectors in mammalian cells.

Expression and cloning vectors should contain a selection gene, also termed a selectable marker. Typically, this is a gene that encodes a protein necessary for the survival or growth of a host cell transformed with the vector. The presence of this gene ensures that any host cell which deletes the vector will not obtain an advantage in growth or reproduction over transformed hosts. Typical selection genes encode proteins that (a) confer resistance to antibiotics or other toxins, e.g. ampicillin, neomycin, methotrexate or tetracycline, (b) complement auxotrophic deficiencies.

Examples of suitable selectable markers for mammalian cells are dihydrofolate reductase (DHFR) or thymidine kinase. Such markers enable the identification of cells which were competent to take up the cerberus nucleic acid. The mammalian cell transformants are placed under selection pressure which only the transformants are uniquely adapted to survive by virtue of having taken up the marker. Selection pressure is imposed by culturing the transformants under conditions in which the concentration of selection agent in the medium is successively changed. Amplification is the process by which genes in greater demand for the production of a protein critical for growth are reiterated in tandem within the chromosomes of successive generations of recombinant cells. Increased quantities of cerberus or frzb-1 can therefor be synthesized from the amplified DNA.

For example, cells transformed with the DHFR selection gene are first identified by culturing all of the transformants in a culture medium which contains methotrexate (Mtx), a competitive antagonist of DHFR. An appropriate host cell in this case is the Chinese hamster ovary (CHO) cell line deficient in DHFR activity, prepared and propagated as described by Urlaub and Chasin, Proc. Nat. Acac. Sci., 77, 4216 (1980). The transformed cells then are exposed to increased levels of Mtx. This leads to the synthesis of multiple copies of the DHFR gene and, concomitantly, multiple copies of other DNA comprising the expression vectors, such as the DNA encoding cerberus or frzb-1. Alternatively, host cells transformed by an expression vector comprising DNA sequences encoding cerberus or frzb-1 and aminoglycoside 3′ phosphotransferase (APH) protein can be selected by cell growth in medium containing an aminoglycosidic antibiotic such as kanamycin or neomycin or G418. Because eukaryotic cells do not normally express an endogenous APH activity, genes encoding APH protein, commonly referred to as neo resistant genes, may be used as dominant selectable markers in a wide range of eukaryotic host cells, by which cells transformed by the vector can readily be identified.

Expression vectors, unlike cloning vectors, should contain a promoter which is recognized by the host organism and is operably linked to the cerberus nucleic acid. Promoters are untranslated sequences located upstream from the start codon of a structural gene (generally within about 100 to 1000 bp) that control the transcription and translation of nucleic acid under their control. They typically fall into two classes, inducible and constitutive. Inducible promoters are promoters that initiate increased levels of transcription from DNA under their control in response to some change in culture conditions, e.g. the presence or absence of a nutrient or a change in temperature. At this time a large number of promoters recognized by a variety of potential host cells are well known. These promoters can be operably linked to cerberus encoding DNA by removing them from their gene of origin by restriction enzyme digestion, followed by insertion 5′ to the start codon for cerberus or frzb-1.

Nucleic acid is operably linked when it is placed into a functional relationship with another nucleic acid sequence. For example, DNA for a presequence or secretory leader is operably linked to DNA for a polypeptide if it is expressed as a preprotein which participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous and, in the case of a secretory leader, contiguous and in reading phase. Linking is accomplished by ligation at convenient restriction sites. If such sites do not exit then synthetic oligonucleotide adapters or linkers are used in accord with conventional practice.

Transcription of the protein-encoding DNA in mammalian host cells is controlled by promoters obtained from the genomes of viruses such as polyoma, cytomegalo-virus, adenovirus, retroviruses, hepatitis-B virus, and most preferably Simian Virus 40 (SV40), or from heterologous mammalian promoters, e.g. the actin promoter. Of course, promoters from the host cell or related species also are useful herein.

Cerberus and frzb-1 are clearly useful as a component of culture media for use in culturing cells, such as endodermal, cardiac, and nerve cells, in vitro. We believe cerberus and frzb-1 will find uses as agents for enhancing the survival or inducing the growth of liver, pancreas, heart, and nerve cells, such as in tissue replacement therapy.

The final cDNA isolated containing a signal sequence results in a peptide designated Paraxial Protocadherin (PAPC). The cDNA for PAPC is a divergent member of the cadherin multigene family. PAPC is most related to protocadherin 43 reported by Sano et al., The EMBO J., 12, pp. 2249-2256, 1993. As shown in SEQ ID NO: 5, the PAPC gene encodes a transmembrane protein of 896 amino acids, of which 187 are part of an intracellular domain. PAPC is a cell adhesion molecule, and microinjection of PAPC mRNA constructs into Xenopus embryos suggest that PAPC acts in mesoderm differentiation. The nucleotide sequence encoding Xenopus PAPC is provided in SEQ ID NO: 6.

Therapeutic formulations of the novel proteins may be prepared for storage by mixing the polypeptides having the desired degree of purity with optional physiologically acceptable carriers, excipients or stabilizers, in the form of lyophilized cake or aqueous solutions. Acceptable carriers, excipients or stabilizers are nontoxic to recipients at the dosages and concentrations employed, and include buffers such as phosphate, citrate, and other organic acids; anti-oxidants including ascorbic acid; low molecular weight (less than about 10 residues) polypeptides; proteins, such as serum albumin, gelatin or immunoglobulins. Other components can include glycine, blutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

Polyclonal antibodies to the novel proteins generally are raised in animals by multiple subcutaneous (sc) or intraperitoneal (ip) injections of cerberus or frzb-1 and an adjuvant. It may be useful to conjugate these proteins or a fragment containing the target amino acid sequence to a protein which is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR.

Animals can be immunized against the immunogenic conjugates or derivatives by combining 1 mg or 1 μg of conjugate (for rabbits or mice, respectively) with 3 volumes of Freund's complete adjuvant and injecting the solution intradermally in multiple sites. One month later the animals are boosted with 1/5 to 1/10 the original amount of conjugate in Fruend's complete adjuvant by subcutaneous injection at multiple sites. Seven to 14 days later animals are bled and the serum is assayed for anti-cerberus titer. Animals are boosted until the titer plateaus. Preferably, the animal is boosted with the conjugate of the same cerberus or frzb-1 polypeptide, but conjugated to a different protein and/or through a different cross-linking agent. Conjugates also can be made in recombinant cell culture as protein fusions. Also, aggregating agents such as alum are used to enhance the immune response.

Monoclonal antibodies are prepared by recovering spleen cells from immunized animals and immortalizing the cells in conventional fashion, e.g. by fusion with myeloma cells or by EB virus transformation and screening for clones expressing the desired antibody.

Antibodies are useful in diagnostic assays for cerberus, frzb-1, or PAPC or their antibodies and to identify family members. In one embodiment of a receptor binding assay, an antibody composition which binds to all of a selected plurality of members of the cerberus family is immobilized on an insoluble matrix, the test sample is contacted with the immobilized antibody composition in order to adsorb all cerberus family members, and then the immobilized family members are contacted with a plurality of antibodies specific for each member, each of the antibodies being individually identifiable as specific for a predetermined family member, as by unique labels such as discrete fluorophores or the like. By determining the presence and/or amount of each unique label, the relative proportion and amount of each family member can be determined.

The antibodies also are useful for the affinity purification of the novel proteins from recombinant cell culture or natural sources. Antibodies that do not detectably cross-react with other growth factors can be used to purify the proteins free from these other family members.

### EXAMPLE 1

### Frzb-1 Antagonizes Xwnt-8 Non-Cell Autonomously

To test whether frzb-1 can antagonize secondary axes caused by Xwnt-8 after secretion by injected cells, an experimental design was used. Thus, frzb-1 mRNA was injected into each of the four animal blastomeres of eight-cell embryos, and subsequently, a single injection of Xwnt-8 mRNA was given to a vegetal-ventral blastomere at the 16-32 cell stage. In two independent experiments, we found that injection of frzb-1 alone (n=13) caused mild dorsalization with enlargement of the cement gland in all embryos and that injection of Xwnt-8 alone (n=53) lead to induction of complete secondary axes in 67% of the embryos. However, injection of frzb-1 into animal caps abolished the formation of complete axes induced by Xwnt-8 (n=27), leaving only a residual 14% of embryos with very weak secondary axes. The double-injected embryos retained the enlarged cement gland phenotype caused by injection of frzb-1 mRNA alone. Because both mRNAs encode secreted proteins and were microinjected into different cells, we conclude that the antagonistic effects of frzb-1 and Xwnt-8 took place in the extracellular space after these proteins were secreted.

### EXAMPLE 2

### Membrane-Anchored Wnt-1 Confers Frzb-1 Binding

To investigate a possible interaction between frzb-1 and Wnts, the first step was to insert an HA epitope tag into a Xenopus frzb-1 construct driven by the CMV (cytomegalovirus) promoter. Frzb1-HA was tested in mRNA microinjection assays in Xenopus embryos and found to be biologically active. Conditioned medium from transiently transfected cells contained up to 10 μg/ml of Frzb1-HA (quantitated on Western blots using an HA-tagged protein standard).

Transient transfection of 293 cells has been instrumental in demonstrating interactions between wingless and frizzled proteins. We therefore took advantage of constructs in which Wnt-1 was fused at the amino terminus of CD8, generating a transmembrane protein containing biologically active Wnt-1 exposed to the extracellular compartment. A Wnt1CD8 cDNA construct (a generous gift of Dr. H. Varmus , NIH) was subcloned into the pcDNA (Invitrogen) vector and transfected into 293 cells. After incubation with Frzb1-HA-conditioned medium (overnight at 37° C.), intensely labeled cells were observed by immunofluorescence. As a negative control, a construct containing 120 amino acids of Xenopus chordin, an unrelated secreted protein was used. Transfection of this construct produced background binding of Frzb1-HA to the extracellular matrix, both uniform and punctate. Cotransfection of Wnt1CD8 with pcDNA-LacZ showed that transfected cells stained positively for Frzb1-HA and LacZ. Since Wnt1CD8 contains the entire CD8 molecule, a CD8 cDNA was used as an additional negative control. After transfection with Lacz and full-length CE8, Frzb1-HA failed to bind to the transfected cells. Although most of our experiments were carried out at 37° C., Frzb1-HA-conditioned medium also stained Wnt1CD8-transfected cells after incubation at 4° C. for 2 hours.

Attempts to biochemically quantitate the binding of Frzb-1 to Wnt1CD8-transfected cells were unsuccessful due to high background binding to control cultures, presumably due to binding to the extracellular matrix. Thus, we were unable to estimate a K_{D} for the affinity of the Frzb-1/Wnt-1 interaction. However, when serial dilutions of conditioned medium containing Frzb1-HA were performed (ranging from 2.5×10⁻⁷ to 1.25×10⁻¹⁰ M), staining of Wnt1CD8-transfected cells was found at all concentrations.

Although we have been unable to provide biochemical evidence for direct binding between Wnts and frzb-1, this cell biological assay indicates that Frzb1-HA can bind, directly or indirectly, to Wnt-1 on the cell membrane in the 10⁻¹⁰ M range.

It is to be understood that while the invention has been described above in conjunction with preferred specific embodiments, the description and examples are intended to illustrate and not limit the scope of the invention, which is defined by the scope of the appended claims.

It is claimed:

1. A substantially pure protein characterized by a physiologically active form and comprising an amino acid sequence encoded by the DNA of SEQ ID NO: 2.

2. The protein as in claim 1 having neurotrophic, growth or differentiation factor activity.

3. A composition comprising the protein of claim 1 and a physiologically acceptable carrier with which the peptide is admixed.

4. An oligonucleotide construct comprising a sequence coding for a protein and an expression vector operatively linked therewith, the protein having neurotrophic, growth or differentiation factor activity and being expressible from SEQ ID NO: 2.

5. The construct as in claim 4 wherein the expression vector is a mammalian or viral expression vector.

6. A substantially pure protein characterized by a physiologically active form and comprising an amino acid sequence encoded by the DNA of SEQ ID NO: 4 1 SEQ ID NO: 8, or SEQ ID NO: 10.

7. The protein as in claim 6 having neurotrophic, growth or differentiation factor activity.

8. A composition comprising the protein of claim 6 and a physiologically acceptable carrier with which the protein is admixed.

9. An oligonucleotide construct comprising a sequence coding for a protein and an expression vector operatively linked therewith, the protein being expressible from SEQ ID NO: 4, SEQ ID NO: 8 or SEQ ID NO:10.

10. The construct as in claim 9 wherein the protein is expressible in soluble form.

11. The construct as in claim 9 wherein the expression vector is a mammalian or viral expression vector.

12. A complex comprising a substantially pure frzb-1 protein complexed with at least one Wnt protein.

13. A substantially pure protein characterized by a physiologically active form and comprising an amino acid sequence encoded by the DNA of SEQ ID NO: 6.

14. The protein as in claim 13 having mesoderm differentiation activity.

15. A composition comprising the protein of claim 13 and a physiologically acceptable carrier with which the protein is admixed.


SEQUENCE LISTING (10 sequences)

SEQ ID NO: 1 - protein, 270 aa, Xenopus
MLLNVLRICIIVCLVNDGAGKHSEGRERTKTYSLNSRGYFRKERGARRSKILLVNTKGLDEPHIGHGDFGLVAELFDSTRTHTNRKEPDMNKVKLFSTVAHGNKSARRKAYNGSRRNIFSRRSFDKRNTEVTEKPGAKMFWNNFLVKMNGAPQNTSHGSKAQEIMKEACKTLPFTQNIVHENCDRMVIQNNLCFGKCISLHVPNQQDRRNTCSHCLPSKFTLNHLTLNCTGSKNVVKVVMMVEECTCEAHKSNFHQTAQFNMDTSTTLHH

SEQ ID NO: 2 - DNA, 1338 bp, Xenopus
gaattcccagcaagtcgctcagaaacactgcagggtctagatatcatacaatgttactaaatgtactcaggatctgtattatcgtctgccttgtgaatgatggagcaggaaaacactcagaaggacgagaaaggacaaaaacatattcacttaacagcagaggttacttcagaaaagaaagaggagcacgtaggagcaagattctgctggtgaatactaaaggtcttgatgaaccccacattgggcatggtgattttcgcttagtagctgaactatttgattccaccagaacacatacaaacagaaaagagccagacatgaacaaagtcaagcttttctcaacagttgcccatggaaacaaaagtgcaagaagaaaagcttacaatggttctagaaggaatatttttcctcgccgttcttttgataaaagaaatacagaggttactgaaaagcctggtgccaagatgttctggaacaattttttggttaaaatgaatggagccccacagaatacaagccatggcagtaaagcacaggaaataatgaaagaagcttgcaaaaccttgtttttcactcagaatattgtacatgaaaactgtgacaggatggtgatacagaacaatctgtgctttggtaaatgcatctctctccatgttccaaatcagcaagatcgacgaaatacttgttcccattgcttgccgtccaaatttaccctgaaccacctgacgctgaattgtactggatctaagaatgtagtaaaggttgtcatgatggtagaggaatgcacgtgtgaagctcataagagcaacttccaccaaactgcacagtttaacatggatacatctactaccctgcaccattaaaggactgccatacagtatggaaatgcccttttgttggaatatttgttacatactatgcatctaaagcattatgttgccttctatttcatataaccacatggaataaggattgtatgaattataattaacaaatggcattttgtgtaacatgcaagatctctgttccatcagttgcaagataaaaggcaatatttgtttgactttttttctacaaaatgaatacccaaatatatgataagataatggggtcaaaactgttaaggggtaatgtaataatagggactaagtttgcccaggagcagtgacccataacaaccaatcagcaggtatgatttactggtcacctgtttaaaagcaaacatcttattggttgctatgggttactgcttctgggcaaaatgtgtgcctcataggggggttagtgtgttgtgtactgaataaattgtatttatttcattgttacaaaaaaaaaaaa

SEQ ID NO: 3 - protein, 318 aa, Xenopus frazzled
MSRTRKVDSLLLLAIPGLALLLLPNAYCASCEPVRIPMCKSMPWNMTKMPNHLHHSTQANAILAIEQFEGLLTTECSQDLLFFLCAMYAPICTIDFQHEPIKPCKSVCERARAGCEPILIKYRHTWPESLACEELPVYDRGVCISPEAIVTVEQGTDSMPDFSMDSNNGNCGSGREHCKCKPMKATQKTYLKNNYNYVIRAKVKEVKVKCHDATAIVEVKEILKSSLVNIPKDTVTLYTNSGCLCPQLVANEEYIIMGYEDKERTRLLLVEGSLAEKWRDRLAKKVKRWDQKLRRPRKSKDPVAPIPNKNSNSRQARS

SEQ ID NO: 4 - DNA, 1875 bp, Xenopus frazzled
gaattccctttcacacaggactcctggcagaggtgaatggttagccctatggatttggtttgttgattttgacacatgattgattgctttcagataggattgaaggacttggatttttatctaattctgcacttttaaattatctgagtaattgttcattttgtattggatgggactaaagataaacttaactccttgcttttgacttgcccataaactataaggtggggtgagttgtagttgcttttacatgtgcccagattttccctgtattccctgtattccctctaaagtaagcctacacatacaggttgggcagaataacaatgtctcgaacaaggaaagtggactcattactgctactggccatacctggactggcgcttctcttattacccaatgcttactgtgcttcgtgtgagcctgtgcggatccccatgtgcaaatctatgccatggaacatgaccaagatgcccaaccatctccaccacagcactcaagccaatgccatcctggcaattgaacagtttgaaggtttgctgaccactgaatgtagccaggaccttttgttctttctgtgtgccatgtatgcccccatttgtaccatcgatttccagcatgaaccaattaagccttgcaagtccgtgtgcgaaagggccagggccggctgtgagcccattctcataaagtaccggcacacttggccagagagcctggcatgtgaagagctgcccgtatatgacagaggagtctgcatctccccagaggctatcgtcacagtggaacaaggaacagattcaatgccagacttctccatggattcaaacaatggaaattgcggaagcggcagggagcactgtaaatgcaagcccatgaaggcaacccaaaagacgtatctcaagaataattacaattatgtaatcagagcaaaagtgaaagaggtgaaagtgaaatgccacgacgcaacagcaattgtggaagtaaaggagattctcaagtcttccctagtgaacattcctaaagacacagtgacactgtacaccaactcaggctgcttgtgcccccagcttgttgccaatgaggaatacataattatgggctatgaagacaaagagcgtaccaggcttctactagtggaaggatccttggccgaaaaatggagagatcgtcttgctaagaaagtcaagcgctgggatcaaaagcttcgacgtcccaggaaaagcaaagaccccgtggctccaattcccaacaaaaacagcaattccagacaagcgcgtagttagactaacggaaaggtgtatggaaactctatggactttgaaactaagatttgcattgttggaagagcaaaaaagaaattgcactacagcacgttatattctattgtttactacaagaagctggtttagttgattgtagttctcctttccttctttttttttataactatatttgcacgtgttcccaggcaattgttttattcaacttccagtgacagagcagtgactgaatgtctcagcctaaagaagctcaattcatttctgatcaactaatggtgacaagtgtttgatacttggggaaagtgaactaattgcaatggtaaatcagagaaaagttgaccaatgttgcttttcctgtagatgaacaagtgagagatcacatttaaatgatgatcactttccatttaatactttcagcagttttagttagatgacatgtaggatgcacctaaatctaaatattttatcataaatgaagagctggtttagactgtatggtcactgttgggaaggtaaatgcctactttgtcaattctgttttaaaaattgcctaaataaatattaagtcctaaataaaaaaaaaaaaaaaaaaaaa

SEQ ID NO: 5 - protein, 896 aa, Xenopus
MLLLFRAIPMLLLGLMVLQTDCEIAQYYIDEEEPPGTVIAVLSQHSIFNTTDIPATNFRLMKQFNNSLIGVRESDGQLSIMERIDREQICRQSLHCNLALDVVSFSKGHFKLLNVKVEVRDINDHSPHFPSEIMHVEVSESSSVGTRIPLEIAIDEDVGSNSIQNFQISNNSHFSIDVLTRADGVKYADLVLMRELDREIQPTYIMELLAMDGGVPSLSGTAVVNIRVLDFNDNSPVFERSTIAVDLVEDAPLGYLLLELHATDDDEGVNGEIVYGFSTLASQEVRQLFKINSRTGSVTLEGQVDFETKQTYEFEVQAQDLGPNPLTATCKVTVHILDVNDNTPAITITPLTTVNAGVAYIPETATKENFIALISTTDRASGSNGQVRCTLYGHEHFKLQQAYEDSYMIVTTSTLDRENIAAYSLTVVAEDLGFPSLKTKKYYTVKVSDENDNAPVFSKPQYEASILENNAPGSYITTVIARDSDSDQNGKVNYRLVDAKVMGQSLTTFVSLDADSGVLRAVRSLDYEKLKQLDFEIEAADNGIPQLSTRVQLNLRIVDQNDNCPVITNPLLNNGSGEVLLPISAPQNYLVFQLKAEDSDEGHNSQLFYTILRDPSRLFAINKESGEVFLKKQLNSDHSEDLSIVVAVYDLGRPSLSTNATVKFILTDSFPSNVEVVILQPSAEEQHQIDMSIIFIAVLAGGCALLLLAIFFVACTCKKKAGEFKQVPEQHGTCNEERLLSTPSPQSVSSSLSQSESCQLSINTESENCSVSSNQEQHQQTGIKHSISVPSYHTSGWHLDNCAMSISGHSHMGHISTKVQWAKEIVTSMTVTLILVENQKRRALSSQCRHKPVLNTQMNQQGSDMPITISATESTRVQKMGTAHCNMKRAIDCLTL

SEQ ID NO: 6 - DNA, 3657 bp, Xenopus
gaattcccagagatgaactccttgagattgttttaaatgactgcaggtctggaaggattcacattgccacactgtttctaggcatgaaaaaactgcaagtttcaactttgtttttggtgcaactttgattcttcaagatgctgcttctcttcagagccattccaatgctgctgttgggactgatggttttacaaacagactgtgaaattgcccagtactacatagatgaagaagaaccccctggcactgtaattgcagtgttgtcacaacactccatatttaacactacagatatacctgcaaccaatttccgtctaatgaagcaatttaataattcccttatcggagtccgtgagagtgatgggcagctgagcatcatggagaggattgaccgggagcaaatctgcaggcagtcccttcactgcaacctggctttggatgtggtcagcttttccaaaggacacttcaagcttctgaacgtgaaagtggaggtgagagacattaatgaccatagccctcactttcccagtgaaataatgcatgtggaggtgtctgaaagttcctctgtgggcaccaggattcctttagaaattgcaatagatgaagatgttgggtccaactccatccagaactttcagatctcaaataatagccacttcagcattgatgtgctaaccagagcagatggggtgaaatatgcagatttagtcttaatgagagaactggacagggaaatccagccaacatacataatggagctactagcaatggatgggggtgtaccatcactatctggtactgcagtggttaacatccgagtcctggactttaatgataacagcccagtgtttgagagaagcaccattgctgtggacctagtagaggatgctcctctgggataccttttgttggagttacatgctactgacgatgatgaaggagtgaatggagaaattgtttatggattcagcactttggcatctcaagaggtacgtcagctatttaaaattaactccagaactggcagtgttactcttgaaggccaagttgattttgagaccaagcagacttacgaatttgaggtacaagcccaagatttgggccccaacccactgactgctacttgtaaagtaactgttcatatacttgatgtaaatgataataccccagccatcactattacccctctgactactgtaaatgcaggagttgcctatattccagaaacagccacaaaggagaactttatagctctgatcagcactactgacagagcctctggatctaatggacaagttcgctgtactctttatggacatgagcactttaaactacagcaagcttatgaggacagttacatgatagttaccacctctactttagacagggaaaacatagcagcgtactctttgacagtagttgcagaagaccttggcttcccctcattgaagaccaaaaagtactacacagtcaaggttagtgatgagaatgacaatgcacctgtattttctaaaccccagtatgaagcttctattctggaaaataatgctccaggctcttatataactacagtgatagccagagactctgatagtgatcaaaatggcaaagtaaattacagacttgtggatgcaaaagtgatgggccagtcactaacaacatttgtttctcttgatgcggactctggagtattgagagctgttaggtctttagactatgaaaaacttaaacaactggattttgaaattgaagctgcagacaatgggatccctcaactctccactcgcgttcaactaaatctcagaatagttgatcaaaatgataattgccctgtgataactaatcctcttcttaataatggctcgggtgaagttctgcttcccatcagcgctcctcaaaactatttagttttccagctcaaagccgaggattcagatgaagggcacaactcccagctgttctataccatactgagagatccaagcagattgtttgccattaacaaagaaagtggtgaagtgttcctgaaaaaacaattaaactctgaccattcagaggacttgagcatagtagttgcagtgtatgacttgggaagaccttcattatccaccaatgctacagttaaattcatcctcaccgactcttttccttctaacgttgaagtcgttattttgcaaccatctgcagaagagcagcaccagatcgatatgtccattatattcattgcagtgctggctggtggttgtgctttgctacttttggccatcttttttgtggcctgtacttgtaaaaagaaagctggtgaatttaagcaggtacctgaacaacacggaacatgcaatgaagaacgcctgttaagcaccccatctccccagtcggtctcttcttctttgtctcagtctgagtcatgccaactctccatcaatactgaatctgagaattgcagcgtgtcctctaaccaagagcagcatcagcaaacaggcataaagcactccatctctgtaccatcttatcacacatctggttggcacctggacaattgtgcaatgagcataagtggacattctcacatggggcacattagtacaaaggtacagtgggcaaaggagatagtgacttcaatgacagtgactctgatactagtggagaatcacaaaagaagagcattgagcagccaatgcaggcacaagccagtgctcaatacacagatgaatcagcagggttccgacatgccgataactatttcagccaccgaatcaacaagggtccagaaaatgggaactgcacattgcaatatgaaaagggctatagactgtcttactctgtagctcctgctcattacaatacctaccatgcaagaatgcctaacctgcacataccgaaccatacccttagagacccttattaccatatcaataatcctgttgctaatcggatgcaggcggaatatgaaagagatttagtcaacagaagtgcaacgttatctccgcagagatcgtctagcagataccaagaattcaattacagtccgcagatatcaagacagcttcatccttcagaaattgctacaaccttttaatcattaggcatgcaagtgagaatgcacaaaggcaagtgctttagcatgaaagctaaatatatggagtctcccctttccctctgatggatggggggagacacaggacagtgcataaatatacagctgctttctatttgcatttcacttgggaattttttgttttttttacatatttatttttcctgaattgaatgtgacattgtcctgtcacctaactagcaattaaatccacagacctacagtcaaatatttgagggcccctgaaacagcacatcagtcaggacctaaagtggcctttttacttttagcagctcctgggtctgccctctgtgttaatcagcccctggtcaagtcctgagtaggatcatggcgtttttatatgcatctcacctactttggacgtgatttacacataataggaaacgcttggtttcagtgaagtctgtgttgtatatattctgttatatacacgcattttgtgtttgtgtatatatttcaagtccattcagatatgtgtatatagtgcagaccttgtaaaattaatattctgatactttttcctcaataaatatttaaat

SEQ ID NO: 7 - protein, 323 aa, Mouse FRZB-1
MVCCGPGRMLLGWAGLLVLAALCLLQVPGAQAAACEPVRIPLCKSLPWNMTKMPNHLHHSTQANAILAMEQFEGLLGTHCSPDLLFFLCAMYAPICTIDFQHEPIKPCKSVCERARQGCEPILIKYRHSWPESLACDELPVYDRGVCISPEAIVTADGADFPMDSSTGHCRGASSERCKCKPVRATQKTYFRNNYNYVIRAKVKEVKMKCHDVTAVVEVKEILKASLVNIPRDTVNLYTTSGCLCPPLTVNEEYVIMGYEDEERSRLLLVEGSIAEKWKDRLGKKVKRWDMKLRHLGLGKTDASDSTQNQKSGRNSNPRPARS

SEQ ID NO: 8 - DNA, 2176 bp, Mouse FRZB-1
aagcctgggaccatggtctgctgcggcccgggacggatgctgctaggatgggccgggttgctagtcctggctgctctctgcctgctccaggtgcccggagctcaggctgcagcctgtgagcctgtccgcatcccgctgtgcaagtcccttccctggaacatgaccaagatgcccaaccacctgcaccacagcacccaggctaacgccatcctggccatggaacagttcgaagggctgctgggcacccactgcagcccggatcttctcttcttcctctgtgcaatgtacgcacccatttgcaccatcgacttccagcacgagcccatcaagccctgcaagtctgtgtgtgagcgcgcccgacagggctgcgagcccattctcatcaagtaccgccactcgtggccggaaagcttggcctgcgacgagctgccggtgtacgaccgcggcgtgtgcatctctcctgaggccatcgtcaccgcggacggagcggattttcctatggattcaagtactggacactgcagaggggcaagcagcgaacgttgcaaatgtaagcctgtcagagctacacagaagacctatttccggaacaattacaactatgtcatccgggctaaagttaaagaggtaaagatgaaatgtcatgatgtgaccgccgttgtggaagtgaaggaaattctaaaggcatcactggtaaacattccaagggacaccgtcaatctttataccacctctggctgcctctgtcctccacttactgtcaatgaggaatatgtcatcatgggctatgaagacgaggaacgttccaggttactcttggtagaaggctctatagctgagaagtggaaggatcggcttggtaagaaagtcaagcgctgggatatgaaactccgacaccttggactgggtaaaactgatgctagcgattccactcagaatcagaagtctggcaggaactctaatccccggccagcacgcagctaaatcctgaaatgtaaaaggccacacccacggactcccttctaagactggcgctggtggactaacaaaggaaaaccgcacagttgtgctcgtgaccgattgtttaccgcagacaccgcgtggctaccgaagttacttccggtcccctttctcctgcttcttaatggcgtggggttagatcctttaatatgttatatattctgtttcatcaatcacgtggggactgttcttttgcaaccagaatagtaaattaaatatgttgatgctaaggtttctgtactggactccctgggtttaatttggtgttctgtaccctgattgagaatgcaatgtttcatgtaaagagagaatcctggtcatatctcaagaactagatattgctgtaagacagcctctgctgctgcgcttatagtcttgtgtttgtatgcctttgtccatttccctcatgctgtgaaagttatacatgtttataaaggtagaacggcattttgaaatcagacactgcacaagcagagtagcccaacaccaggaagcatttatgaggaaacgccacacagcatgacttattttcaagattggcaggcagcaaaataaatagtgttgggagccaagaaaagaatattttgcctggttaaggggcacactggaatcagtagcccttgagccattaacagcagtgttcttctggcaagtttttgatttgttcataaatgtattcacgagcattagagatgaacttataactagacatctgttgttatctctatagctctgcttccttctaaatcaaacccattgttggatgctccctctccattcataaataaatttggcttgctgtattggccaggaaaagaaagtattaaagtatgcatgcatgtgcaccagggtgttatttaacagaggtatgtaactctataaaagactataatttacaggacacggaaatgtgcacatttgtttactttttttcttccttttgctttgggcttgtgattttggtttttggtgtgtttatgtctgtattttggggggtgggtaggtttaagccattgcacattcaagttgaactagattagagtagactaggctcattggcctagacattatgatttgaatttgtgttgtttaatgctccatcaagatgtctaataaaaggaatatggttgtcaacagagacgacaacaacaacaaa

SEQ ID NO: 9 - protein, 325 aa, Human FRZB-1
MVCGSPGGMLLLRAGLLALAALCLLRVPGARAAACEPVRIPLCKSLPWNMTKMPNHLHHSTQANAILAIEQFEGLLGTHCSPDLLFFLCAMYAPICTIDFQHEPIKPCKSVCERARQGCEPILIKYRHSWPENLACEELPVYDRGVCISPEAIVTADGADFPMDSSNGNCRGASSERCKCKPIRATQKTYFRNNYNYVIRAKVKEIKTKCHDVTAVVEVKEILKSSLVNIPRDTVNLYTSSGCLCPPLNVNEEYIIMGYEDEERSRLLLVEGSIAEKWKDRLGKKVKRWDMKLRHLGLSKSDSSNSDSTQSQKSGRNSNPRQARN

SEQ ID NO: 10 - DNA, 1893 bp, Human FRZB-1
ggcggagcgggccttttggcgtccactgcgcggctgcaccctgccccatctgccgggatcatggtctgcggcagcccgggagggatgctgctgctgcgggccgggctgcttgccctggctgctctctgcctgctccgggtgcccggggctcgggctgcagcctgtgagcccgtccgcatccccctgtgcaagtccctgccctggaacatgactaagatgcccaaccacctgcaccacagcactcaggccaacgccatcctggccatcgagcagttcgaaggtctgctgggcacccactgcagccccgatctgctcttcttcctctgtgccatgtacgcgcccatctgcaccattgacttccagcacgagcccatcaagccctgtaagtctgtgtgcgagcgggcccggcagggctgtgagcccatactcatcaagtaccgccactcgtggccggagaacctggcctgcgaggagctgccagtgtacgacaggggcgtgtgcatctctcccgaggccatcgttactgcggacggagctgattttcctatggattctagtaacggaaactgtagaggggcaagcagtgaacgctgtaaatgtaagcctattagagctacacagaagacctatttccggaacaattacaactatgtcattcgggctaaagttaaagagataaagactaagtgccatgatgtgactgcagtagtggaggtgaaggagattctaaagtcctctctggtaaacattccacgggacactgtcaacctctataccagctctggctgcctctgccctccacttaatgttaatgaggaatatatcatcatgggctatgaagatgaggaacgttccagattactcttggtggaaggctctatagctgagaagtggaaggatcgactcggtaaaaaagttaagcgctgggatatgaagcttcgtcatcttggactcagtaaaagtgattctagcaatagtgattccactcagagtcagaagtctggcaggaactcgaacccccggcaagcacgcaactaaatcccgaaatacaaaaagtaacacagtggacttcctattaagacttacttgcattgctggactagcaaaggaaaattgcactattgcacatcatattctattgtttactataaaaatcatgtgataactgattattacttctgtttctcttttggtttctgcttctctcttctctcaacccctttgtaatggtttgggggcagactcttaagtatattgtgagttttctatttcactaatcatgagaaaaactgttcttttgcaataataataaattaaacatgctgttaccagagcctctttgctgagtctccagatgttaatttactttctgcaccccaattgggaatgcaatattggatgaaaagagaggtttctggtattcacagaaagctagatatgccttaaaacatactctgccgatctaattacagccttatttttgtatgccttttgggcattctcctcatgcttagaaagttccaaatgtttataaaggtaaaatggcagtttgaagtcaaatgtcacataggcaaagcaatcaagcaccaggaagtgtttatgaggaaacaacacccaagatgaattatttttgagactgtcaggaagtaaaataaataggagcttaagaaagaacattttgcctgattgagaagcacaactgaaaccagtagccgctggggtgttaatggtagcattcttcttttggcaatacatttgatttgttcatgaatatattaatcagcattagagaaatgaattataactagacatctgctgttatcaccatagttttgtttaatttgcttccttttaaataaacccattggtgaaagtcaaaaaaaaaaaaaaaaa